(19) United States
(12) Patent Application Publication
(10) Pub. No.: US 2023/0200439 A1
(43) Pub. Date: Jun. 29, 2023

(54) SEALING MEMBER, LEAK-PROOF ATOMIZER, AND INHALATION DEVICE

(71) Applicant: SHENZHEN ZUN YI PIN TECHNOLOGY CO., LTD., Shenzhen (CN)

(72) Inventors: YING YANG, Shenzhen (CN); MIAOWEN YANG, Shenzhen (CN); SHIMEI PAN, Shenzhen (CN); YONGHUI LIN, Shenzhen (CN); FENG WANG, Shenzhen (CN)

(21) Appl. No.: 17/709,694

(22) Filed: Mar. 31, 2022

Related U.S. Application Data
(63) Continuation-in-part of application No. PCT/CN2021/142543, filed on Dec. 29, 2021.

(51) Int. Cl. A24F 40/42; A24F 40/10; A24F 40/485; A24F 40/46; A61M 11/08; A61M 11/04

(52) U.S. Cl. CPC A24F 40/42; A24F 40/10; A24F 40/485; A24F 40/46; A61M 11/08; A61M 11/041

(57) ABSTRACT

A sealing member for sealing an atomizer against undesired leakage of liquid includes a base body and a resisting portion. The base body includes a first surface, a second surface opposite to the first surface, and an outer wall connecting the first surface with the second surface. A vent passes through the first and second surfaces. The resisting portion is disposed on the outer wall and is configured to be compressed against an inner wall of a liquid storage pipe to provide sealing and avoid leakage. An atomizer and an inhalation device are also disclosed.

### FIELD

The subject matter relates to the field of loading a liquid atomizable substance, after being atomized, into a human body, in particular to an atomizer and an inhalation device including the atomizer.

### BACKGROUND

An inhalation device generally incudes a liquid storage member, an atomizer, and a power supply assembly. The atomizer heats a liquid atomizable substance to generate aerosol by means of atomization for a user to inhale. The power supply assembly is used to supply power to the atomizer. The liquid storage member includes a liquid storage cotton, a liquid storage pipe, and a sealing plug. The liquid storage cotton is disposed in the liquid storage pipe. The liquid atomizable substance absorbed on the liquid storage cotton is provided to the atomizer, and the sealing plug is disposed on an end of the liquid storage pipe to avoid liquid leakage.

However, when the inhalation device is not in use and being carried, the liquid atomizable substance in the liquid storage pipe may leak and pollute the power supply assembly due to poor sealing between the sealing plug and the liquid storage pipe.

### BRIEF DESCRIPTION OF THE DRAWINGS

FIG. 1 is a perspective view of an atomizer according to an embodiment of the present disclosure.

FIG. 2 is an exploded view of the atomizer of FIG. 1.

FIG. 3 is an exploded view of an atomizing assembly of the atomizer of FIG. 2.

FIG. 4 is a cross-sectional view along line IV-IV of FIG. 1 with a sealing member removed.

FIG. 5 is an exploded view of the atomizer of FIG. 1 with a liquid storage pipe removed.

FIG. 6 is a cross-sectional view along line VI-VI of FIG. 1.

FIG. 7 is an exploded view of the atomizer of FIG. 1 with the liquid storage pipe removed from another perspective.

FIG. 8 is a perspective view of an inhalation device according to an embodiment of the present disclosure.

FIG. 9 is an exploded view of the inhalation device of FIG. 8.

FIG. 10 is a cross-sectional view along line X-X of FIG. 8.

FIG. 11 is a schematic structural diagram of a sealing member of an atomizer according to a second embodiment of the present disclosure.

FIG. 12 is a schematic structural diagram of a sealing member of an atomizer according to a third embodiment of the present disclosure.

### DETAILED DESCRIPTION

The present technology in the application will now be described by way of embodiments as follows. Obviously, the described embodiments are a portion of the embodiments of the application, not all of them. Unless otherwise defined, all technical and scientific terms herein have the same meanings as those commonly understood by those skilled in the art. The terms used in the detail description are only for describing specific embodiments, but not intended to limit the present application.

Hereinafter, embodiments of the present application will be described in detail. However, the present application may be embodied in many different forms, and should not be construed as limited to the exemplary embodiments explained herein. Rather, these exemplary embodiments are provided so that the present application can be clearly and specifically conveyed to those skilled in the art.

In addition, for simplicity and clarity, the size or thickness of various components and layers may be enlarged in the drawings. Throughout the whole application, the same symbols refer to the same elements. As used herein, the terms “and/or” include any combination of one or more related items. In addition, it should be understood that when an element A is referred to as “being connected” to an element B, the element A may be directly connected to the element B, or there may be an intermediate element C, so that the element A and the element B may be indirectly connected to each other by the element C.

Furthermore, when describing the embodiments of the present application, “may” signifies that one or more embodiments of the present application is or are being referred to.

The technical terms used herein are for describing specific embodiments, and are not intended to limit the present application. As used herein, the singular form is intended to include the plural form, unless the context expressly indicates otherwise. It should be further understood that the term “including”, when used in this specification, refers to the existence of the described features, values, steps, operations, elements and/or components, but does not exclude the existence or addition of one or more other features, values, steps, operations, elements, components and/or combinations thereof.

Terms related to spatial relationships, such as “on”, can be used in this application for convenient description to describe the relationship between one element or feature and another element (multiple elements) or feature (multiple features) shown in the drawings. It should be understood that in addition to the directions shown in the drawings, the terms related to space are also intended to include different directions of the equipment or devices in use or in operation. For example, if the equipment in the drawings is flipped over, the features described as “above” or “on” other elements or features will be then “below” or “under” other elements or features. Therefore, the terms “on” may include upper and lower directions. It should be understood that although the terms first, second, third, etc. can be used herein to describe various elements, components, regions, layers and/or parts, these elements, components, regions, layers and/or parts should not be limited. These terms are used to distinguish one element, component, region, layer, or part from another element, component, region, layer, or part. Therefore, a first element, component, region, layer, or part discussed below may also be referred to as a second element, component, region, layer, or part within the principles of the present embodiments.

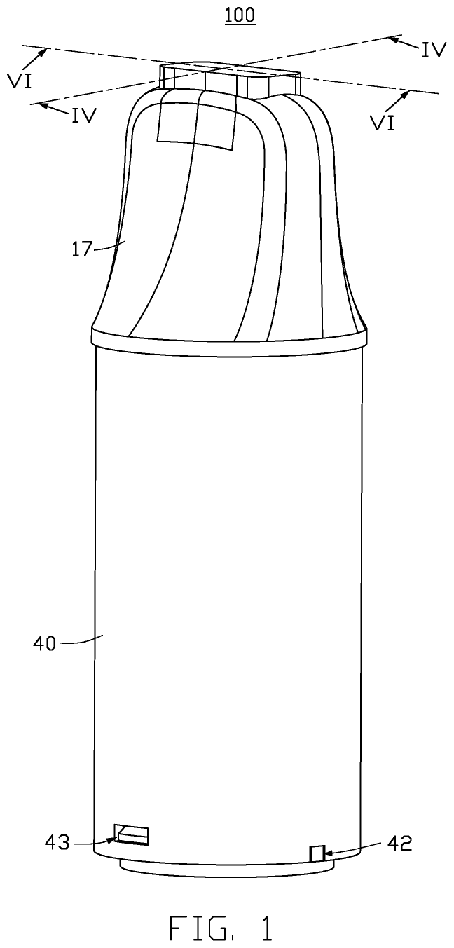

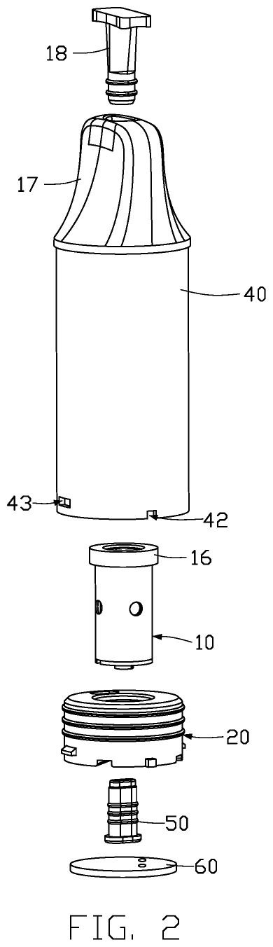

Referring to FIGS. 1 and 2, an embodiment of an atomizer 100 is provided. The atomizer 100 includes an atomizing assembly 10, a sealing member 20, and a liquid storage pipe 40. A liquid atomizable substance is stored in the liquid storage pipe 40. The atomizing assembly 10 is accommodated in the liquid storage pipe 40. The sealing member 20 is disposed on an end of the liquid storage pipe 40 to avoid leakage of the liquid atomizable substance stored in the liquid storage pipe 40.

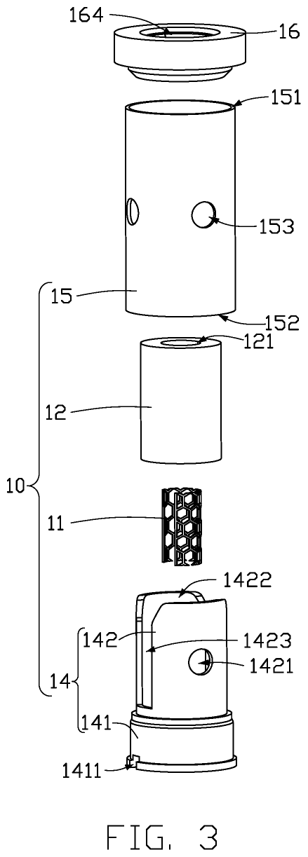

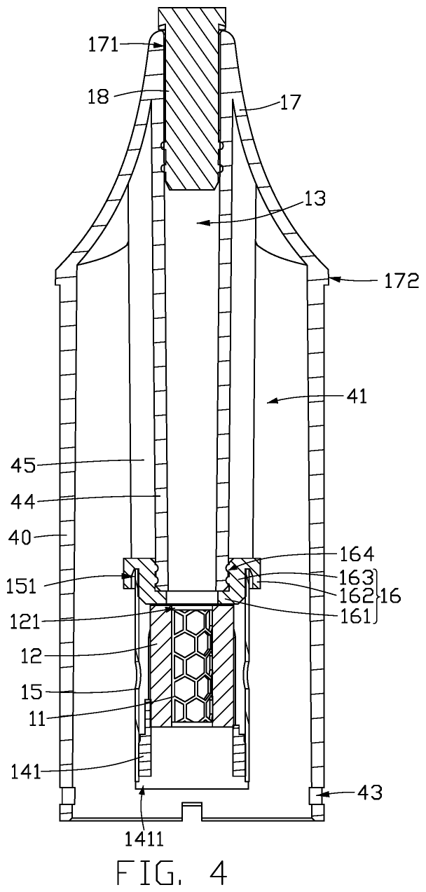

Referring to FIGS. 3 and 4, the atomizing assembly 10 includes a heating member 11, a liquid guiding member 12, a core shell 14, and a liquid guiding pipe 15. The heating member 11 and the liquid guiding member 12 are disposed in the core shell 14. The liquid guiding pipe 15 is sleeved on the core shell 14 and configured to transport the liquid atomizable substance stored in the liquid storage pipe 40 to the liquid guiding member 12. The liquid atomizable substance includes a target active ingredient. An air guiding pipe 44 is also disposed in the liquid storage pipe 40. An end of the liquid guiding pipe 15 communicates with the air guiding pipe 44. The heating member 11 is used to heat the liquid atomizable substance to obtain an aerosol. The air guiding pipe 44 is used to guide the aerosol out for a user to inhale.

Referring to FIGS. 3 and 4, in some embodiments, an end of the liquid storage pipe 40 is provided with a suction nozzle 17. The liquid storage pipe 40 and the suction nozzle 17 are formed as an integral unit. The suction nozzle 17 includes a suction port 171 and an air passage 13 communicating with the suction port 171. The suction port 171 communicates with the air guiding pipe 44 through the air passage 13. The air guiding pipe 44, the suction port 171, and the liquid storage pipe 40 may be arranged coaxially to facilitate egress of the aerosol generated by the atomizing assembly 10, guiding it along the air passage 13 to the suction port 171, for the user to inhale. In some embodiments, the shape of the suction nozzle 17 may be ergonomically appropriate, such as round, oval, or duck-billed.

In some embodiments, the suction nozzle 17 is provided with a dust plug 18 therein. The dust plug 18 is detachably disposed in the suction port 171 to prevent external dust or water vapor from ingress into the suction port 171 when the atomizer 100 is not in use.

Referring to FIGS. 3 and 4, the liquid guiding member 12 includes a first through hole 121 and the heating member 11 is disposed in the first through hole 121. The heating member 11 is in contact with an inner wall of the first through hole 121, so that the heating member 11 can fully atomize the liquid atomizable substance in the liquid guiding member 12. In some embodiments, the heating member 11 may be net-like in shape. Compared with a single heating wire, a larger contact area can be formed between the netlike heating member and the liquid atomizable substance, which makes the heating more uniform. Moreover, compared with the single heating wire, the netlike heating member has a smaller change of resistance during temperature changes. In some specific embodiments, the netlike heating member may be made of stainless steel. In some embodiments, the liquid guiding member 12 may be made of cotton material or a polymer material with adsorption properties.

Referring to FIGS. 3 and 4, a cross section of the core shell 14 can be substantially circular and define a receiving cavity 1422. The core shell 14 includes a base 141 and a housing 142 disposed on the base 141. The housing 142 defines a slot 1423 communicating with the receiving cavity 1422. The housing 142 also defines an infusion hole 1421. The liquid guiding member 12 with the heating member 11 installed is accommodated in the core shell 14, and the liquid atomizable substance stored in the liquid storage pipe 40 can be continuously transported to the liquid guiding member 12 through the slot 1423 and the infusion hole 1421. The entirety of the core shell 14 is accommodated in the liquid guiding pipe 15, and the base 141 abuts against an inner wall of the liquid guiding pipe 15, so that the liquid guiding pipe 15 serves as an integral packaging of the atomizing assembly 10.

In some embodiments, the liquid guiding pipe 15 includes a first end 151 and a second end 152 opposite to the first end 151. The first end 151 of the liquid guiding pipe 15 communicates with the air guiding pipe 44 in the liquid storage pipe 40. The first through hole 121 of the liquid guiding member 12 is arranged coaxially with the air guiding pipe 44, and an inner diameter of the air guiding pipe 44 is substantially the same as that of the first through hole 121 in the liquid guiding member 12, so that the aerosol generated by the heating element 11 when heating the liquid atomizable substance is directly supplied to the user to inhale through the air passage 13. In the exemplary embodiment, two symmetrical slots 1423 are defined on the housing 142, allowing convenient installation of the liquid guiding member 12 in the core shell 14. In some embodiments, the core shell 14 may be made of polycarbonate which can resist strong impacts.

Referring to FIGS. 3 and 4, a second sealing body 16 is disposed between the air guiding pipe 44 and the liquid guiding pipe 15. The second sealing body 16 is used to seal a gap between the first end 151 of the liquid guiding pipe 15 and the air guiding pipe 44 to prevent the liquid atomizable substance stored in the liquid storage pipe 40 from penetrating into the gap between the liquid guiding pipe 15 and the air guiding pipe 44. The second sealing body 16 defines a second through hole 164, the second through hole 164 enables communication between the first through hole 121 and the air passage 13. The second sealing body 16 includes a sealing portion 161, a package portion 162, and a connecting portion 163 connected to the sealing portion 161 and the package portion 162. The sealing portion 161 and the connecting portion 163 are located in the liquid guiding pipe 15, and the package portion 162 is disposed on an outer wall of the first end 151 of the liquid guiding pipe 15. The package portion 162 and the connecting portion 163 of the second sealing body 16 cooperatively form a mounting portion sleeved on an inner wall and the outer wall of the first end 151. The second through hole 164 is located in the middle of the connecting portion 163, the second sealing body 16 extends towards a central axis of the second through hole 164 at an inner wall of the second through hole 164 to form the sealing portion 161. The sealing portion 161 abuts against a top surface of the liquid guiding member 12 to limit a position of the liquid guiding pipe 15 in the liquid guiding member 12 and to prevent the liquid guiding member 12 from moving in the core shell 14.

Referring to FIGS. 3 and 4, a bottom end of the air guiding pipe 44 is embedded in the second through hole 164 of the second sealing body 16 and abuts against the second sealing body 16. The inner wall of the second through hole 164 is provided with threads which abut against an outer wall of the air guiding pipe 44. In some specific embodiments, the second sealing body 16 is made of silica gel, so that the threads of the second sealing body 16 are compressed against the outer wall of the air guiding pipe 44 to prevent the liquid atomizable substance stored in the liquid storage pipe 40 from penetrating into the air passage 13 at the connection between the air guiding pipe 44 and the second through hole 164. A resisting plate 45 is disposed on the outer wall of the air guiding pipe 44, and an end of the resisting plate 45 abuts against the connection between the package portion 162 and the connecting portion 163, so as to prevent an end of the air guiding pipe 44 extending too far into the liquid guiding pipe 15 and squeezing the second sealing body 16, resulting in excessive deformation of the second sealing body 16 that causes liquid leakage, when the atomizing assembly 10 is installed with the air guiding pipe 44. In some embodiments, a plurality of resisting plates 45 may be uniformly arranged in a circumferential direction of the liquid guiding pipe 15 to ensure that the force of all the resisting plates 45 on the second sealing body 16 is balanced, so as to avoid local excessive deformation due to an unbalanced force acting on the second sealing body 16, which would affect the tightness between the air guiding pipe 44 and the liquid guiding pipe 15. In the exemplary embodiment, two resisting plates 45 are symmetrically arranged along a central axis of the air guiding pipe 44.

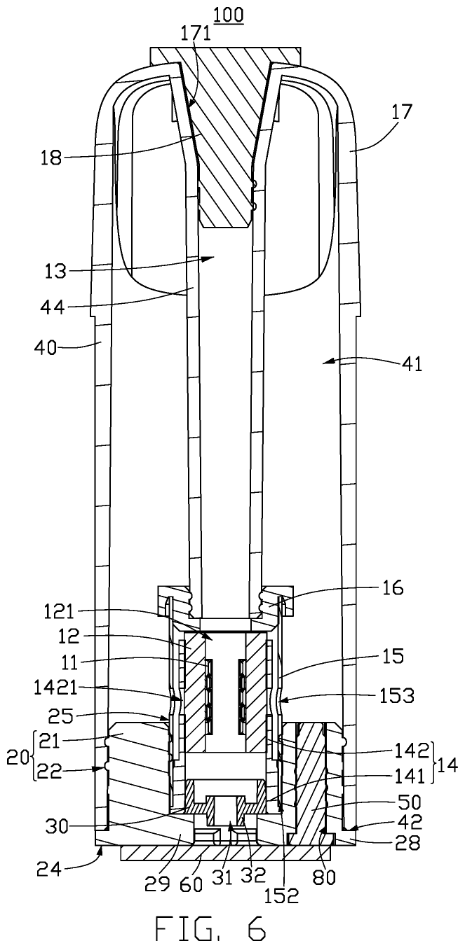

Referring to FIGS. 2 to 4, in some embodiments, an end of the air guiding pipe 44 is fixed to the suction nozzle 17, and the other end of the air guiding pipe 44 extends from the suction port 171 of the suction nozzle 17 and is connected to the liquid guiding pipe 15. The suction nozzle 17 includes a cavity communicating with the liquid storage pipe 40. The cavity of the suction nozzle 17, the inner wall of the liquid storage pipe 40, the outer wall of the air guiding pipe 44, and the outer wall of the liquid guiding pipe 15 form a liquid storage chamber 41. The liquid guiding pipe 15 defines a plurality of liquid guiding holes 153, each corresponding to the slots 1423 of the core shell 14 and the infusion hole 1421 (shown in FIG. 6). The liquid storage chamber 41 communicates with the liquid guiding member 12 in the core shell 14 through the liquid guiding holes 153, so that the liquid atomizable substance in the liquid storage chamber 14 can be continuously transported to the liquid guiding member 12.

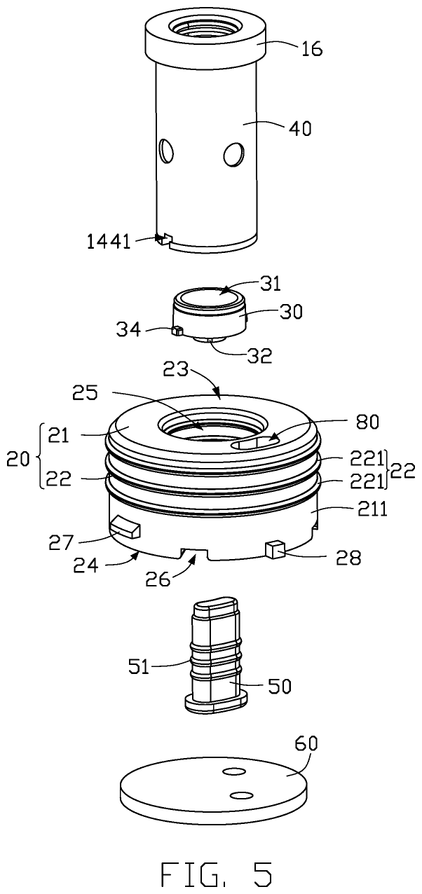

Referring to FIGS. 5 and 6, the sealing member 20 is accommodated in an end of the liquid storage pipe 40 away from the suction nozzle 17 and is flush with the end of the liquid storage pipe 40. The liquid atomizable substance in the liquid storage chamber 14 is sealed in the liquid storage pipe 40 by the sealing member 20. The sealing member 20 includes a base body 21 and a resisting portion 22. The base body 21 is substantially cylindrical and defines a vent 25 which passes through the base body 21 and corresponds to the first through hole 121 of the liquid guiding member 12. When the user draws from the suction port 171, the outside air enters the first through hole 121 from the vent 25 and carries the aerosol through the air passage 13 for the user to inhale.

Referring to FIGS. 5 and 6, along a direction of a central axis of the vent 25, the base body 21 includes a first surface 23, a second surface 24 opposite to the first surface 23, and an outer wall 211 connecting the first surface 23 and the second surface 24. The resisting portion 22 is disposed on the outer wall 211 of the base body 21. The resisting portion 22 includes a plurality of sealing rings 221 disposed annularly on the outer wall 211. The sealing rings 221 are slightly compressed against the inner wall of the liquid storage pipe 40, so that the resisting portion 22 of the sealing member 20 presses tightly against the inner wall of the liquid storage pipe 40, preventing the liquid atomizable substance in the liquid storage chamber 41 from leaking from a gap between the liquid storage pipe 40 and the base body 21. In some embodiments, the number of the sealing rings 221 can be set as needed. In the embodiment, the resisting portion 22 includes a plurality of sealing rings 221 disposed circumferentially around the base body 21, the sealing rings 221 are spaced from each other. As an example, there may be three sealing rings 221. The outer wall 211 is also provided with a plurality of mounting protrusions 27, the mounting protrusions 27 abut against the second surface 24 of the base body 21. The liquid storage pipe 40 defines a plurality of mounting holes 43 (shown in FIG. 4) corresponding to the mounting protrusions 27. The sealing member 20 is fixed in the mounting holes 43 of the liquid storage pipe 40 by the mounting protrusions 27. Even when the atomizer 100 is just being carried, the base body 21 and the liquid storage pipe 40 remain tightly sealed, the tightness of the sealing member 20 in sealing the liquid storage pipe 40 is improved. In some embodiments, the base body 21 is provided with two mounting protrusions 27 opposite to each other.

In some embodiments, the outer wall 211 is also provided with two alignment protrusions 28, the inner wall of the liquid storage pipe 40 defines two alignment notches 42. The two alignment notches 42 correspond to the alignment protrusions 28 of the base body 21. When the sealing member 20 is mounted to the liquid storage pipe 40, the matching of the alignment protrusions 28 and the alignment notches 42 allows rapid and convenient installation of the sealing member 20 in the liquid storage pipe 40. In one embodiment, the alignment notches 42 penetrate a wall of the liquid storage pipe 40.

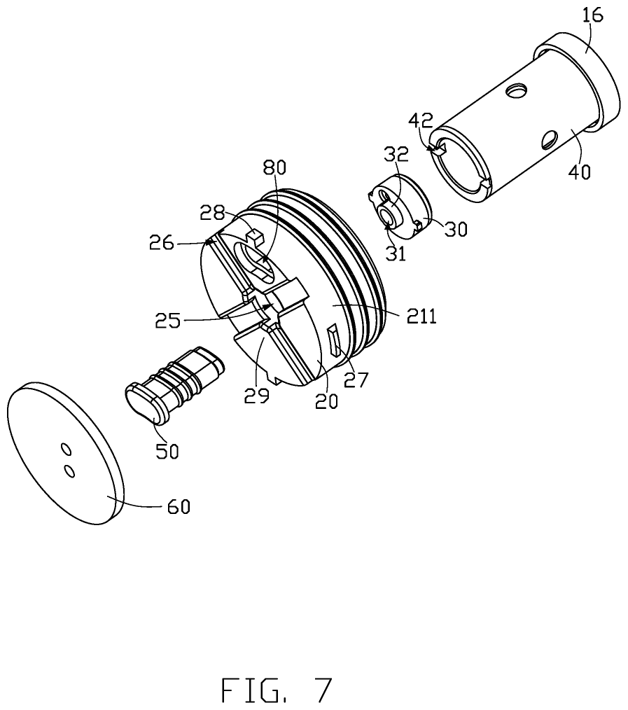

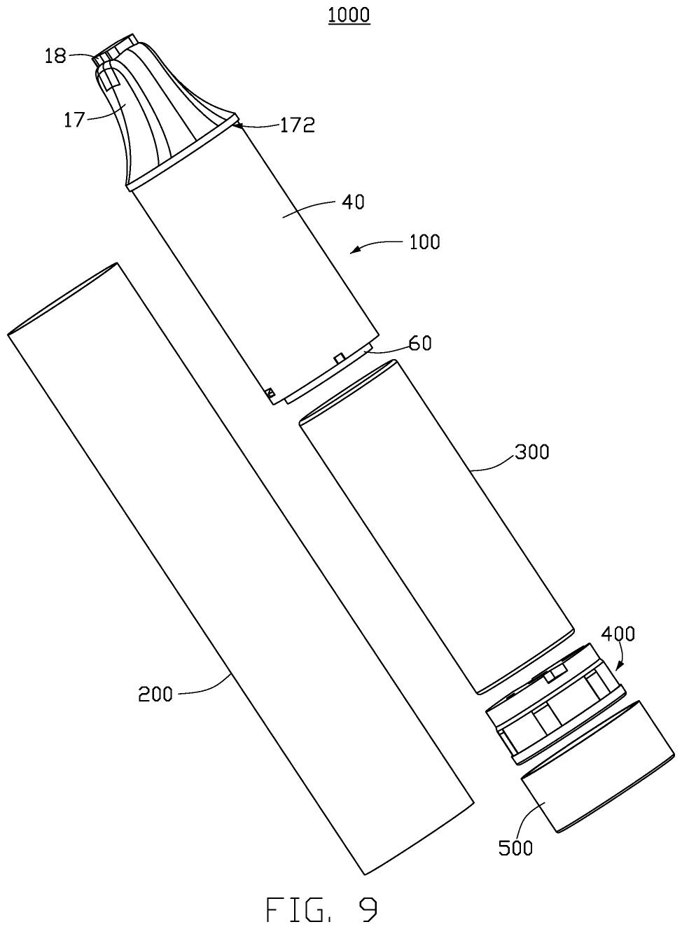

In some embodiments, referring to FIGS. 5 and 7, the second surface 24 of the base body 21 also defines a plurality of air ducts 26 communicating with the vent 25. The air ducts 26, in various shapes, may be arranged along a radial direction of the base body 21, the shapes may be a straight line, Y-shaped, or cross shaped, and communicate with the vent 25. That is, the air ducts 26 penetrate the second surface 24 to the outer wall 211. The outside air flows into the liquid guiding member 12 through the air ducts 26 and the vent 25. As an example, the air ducts 26 are arranged to form a cross shape. The air ducts 26 are arranged on the base body 21 at equal intervals, so that the outside air enters evenly and dispersedly into the air ducts 26. Compared with an existing way of intaking air through the vent 25, this arrangement can prevent the liquid atomizable substance in the liquid storage pipe 40 from directly and vertically dripping on a power supply assembly 300 connected with the sealing member 20 through the vent 25 (see FIG. 9), thereby reducing the harm caused by liquid leakage. In addition, through the arrangement of the air ducts 26, the material of the sealing member 20 is reduced, and the production cost of the sealing member 20 is reduced. In some embodiments, the sealing member 20 is made of silica gel.

Referring to FIGS. 6 and 7, part of an inner wall of the vent 25 of the sealing member 20 extends towards a central axis of the vent 25 to form a flange 29, which is close to the second surface 24 of the base body 21. A bottom of the liquid guiding pipe 15 equipped with the core shell 14 extends into the vent 25 and abuts against the flange 29. The inner wall of the vent 25 is provided with threads to improve sealing between the liquid guiding pipe 15 and the sealing member 20.

Referring to FIGS. 5 to 7, in some embodiments, a first sealing body 30 is disposed between the atomizing assembly 10 and the sealing member 20, the first sealing body 30 is used to seal a gap between the atomizing assembly 10 and the sealing member 20. The first sealing body 30 includes a through hole 31 with a central axis which is collinear with a central axis of the vent 25, the through hole 31 enables communication between the vent 25 and the first through hole 121. A bottom of the first sealing body 30 is placed on the flange 29 and is located in the base 141 of the core shell 14. The first sealing body 30 extends in the direction of the central axis of the through hole 31 to form a guide pipe 32, guiding the outside air flowing into the first through hole 121. The outer wall of the first sealing body 30 abuts against the inner wall of the base 141 to prevent the liquid atomizable substance in the liquid storage pipe 40 from leaking from the connection between the core shell 14 and the liquid guiding pipe 15. The heating member 11 abuts against the first sealing body 30 through a fixing member (not shown) to avoid deformation of the heating member 11, so that the heating member 11 is always in full contact with the liquid guiding member 12. That is, there is enough of a contact area between the heating member 11 and the liquid guiding member 12, so that the heating member 11 can produce enough aerosol after heating the liquid atomizable substance in the liquid guiding member 12. A plurality of clamping blocks 34 are arranged on the outer wall of the first sealing body 30, and the core shell 14 is provided with a plurality of clamping notches 1411 corresponding to the clamping blocks 34. In the embodiment, two opposite clamping blocks 34 are arranged so that the core shell 14 can be accurately and quickly installed on the first sealing body 30.

Referring to FIGS. 6 and 7, the sealing member 20 is installed at an end of the liquid guiding pipe 15 to seal the liquid atomizable substance stored in the liquid storage chamber 41 in the liquid storage pipe 40. The inner wall of the vent 25 of the sealing member 20 is tightly sleeved on the second end 152 of the liquid guiding pipe 15 to prevent the liquid atomizable substance in the liquid storage chamber 41 from leaking from the gap between the sealing member 20 and the liquid guiding pipe 15. Further, the base 141 of the core shell 14 is tightly attached to the inner wall of the liquid guiding pipe 15, and the core shell 14 is tightly attached to the outer wall of the first sealing body 30, preventing the liquid atomizable substance from leaking from the gap between the liquid guiding pipe 15 and the core shell 14. In this case, the liquid atomizable substance in the liquid storage chamber 41 is always sealed in the liquid storage pipe 40 by the sealing member 20, so as to improve the sealing tightness of the liquid storage pipe 40 in sealing the liquid atomizable substance.

Referring to FIGS. 6 and 7, the sealing member 20 also defines a liquid injection port 80, which is spaced from the vent 25, and a plug 50 is movably arranged in the liquid injection port 80. When injecting liquid into the liquid storage pipe 40, the plug 50 is removed, the liquid atomizable substance is injected into the liquid storage chamber 41 through a liquid injection gun or other liquid injector, and then the plug 50 is inserted into the liquid injection port 80. In some embodiments, the plug 50 is made of silica gel, an outer wall of the plug 50 is provided with a protrusion 51, and the protrusion 51 is disposed annularly on the plug 50, improving the sealing between the plug 50 and the sealing member 20.

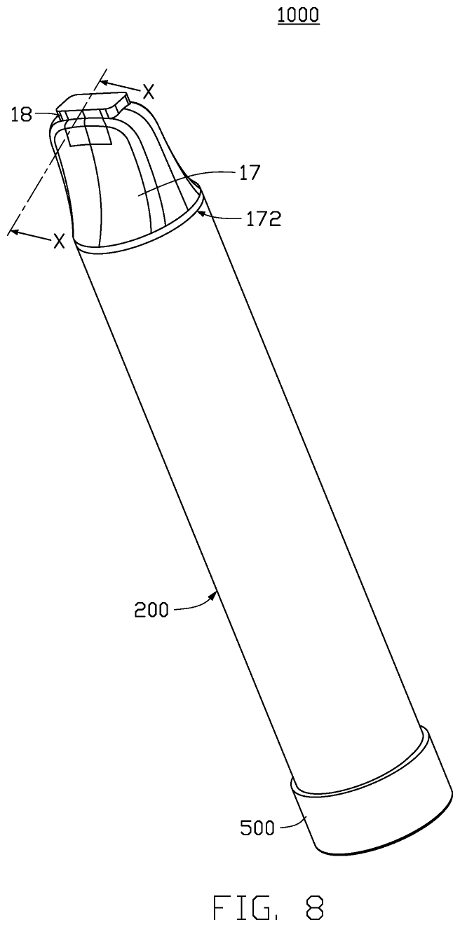

Referring to FIGS. 8 and 9, an embodiment of an inhalation device 1000 is provided, the inhalation device 1000 includes a power supply assembly 300, an outer casing 200, a mounting member 400, and the atomizer 100. The power supply assembly 300 is disposed on an end of the liquid storage pipe 40 away from the suction nozzle 17. A liquid absorbent cotton 60 is disposed between the sealing member 20 and the power supply assembly 300, and the liquid absorbent cotton 60 adsorbs any liquid atomizable substance leaking from the atomizer 100, to avoid polluting the power supply assembly 300 and reducing the safety risk to the power supply assembly 300.

In some embodiments, the inhalation device 1000 may be an electronic cigarette, that is, the liquid atomizable substance is e-liquid. In other embodiments, the inhalation device 1000 is not limited to being an electronic cigarette, but may also be other device for atomizing the liquid atomizable substance into aerosol for the user to inhale. For example, the inhalation device 1000 may be an instrument conveying the aerosol formed by atomizing, such as a medical atomization inhaler for treating upper respiratory tract diseases. In this case, the liquid atomizable substance is a liquid drug for treating upper respiratory tract diseases. A patient can inhale drug in the form of a mist, formed by atomizing the drug liquid, into the respiratory tract and the alveoli through breathing, thus carrying out local drug treatment of the upper respiratory tract. The inhalation device 1000 can be used to convey the aerosol formed by atomizing the liquid atomizable substance onto surface of the skin. For example, the aerosol can be applied onto the skin for cosmetic purposes by the inhalation device 1000.

Compared with intravenous injection and oral administration by swallowing, a manner of atomizing drug liquid into an aerosol allows the aerosol to directly act on a required treatment location, achieving better treatment effect. In an intravenous injection, the drug liquid is injected into the vein through a syringe and finally transported to the required treatment location. The drug liquid goes through blood circulation in the intravenous injection process. In the oral administration by swallowing, the drug liquid passes through the digestive system and goes through blood circulation to reach the required treatment location. The two manners of intravenous injection and oral administration do not allow immediate and full activation of the drug on the required treatment location. In addition, on some occasions, compared with the oral administration, the manner of atomizing drug liquid is more convenient.

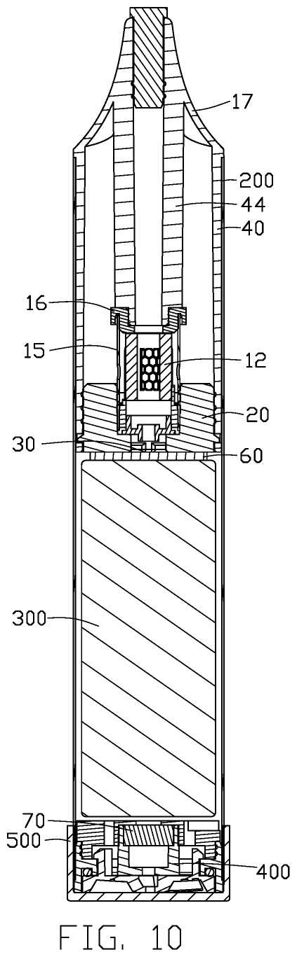

Referring to FIGS. 9 and 10, a bulge loop 172 is disposed at the connection between the suction nozzle 17 and the liquid storage pipe 40. The outer casing 200 is sleeved on the liquid storage pipe 40, and an end of the outer casing 200 abuts against the bulge loop 172. The outer casing 200 is fixed to the outer wall of the liquid storage pipe 40 by an adhesive (not shown). In other embodiments, the outer casing 200 and the suction nozzle 17 may be fixed to each other by means of magnetism. The power supply assembly 300 is accommodated in the outer casing 200 and is fixed in the outer casing 200 by the mounting member 400. In some embodiments, the power supply assembly 300 may be a cylindrical battery.

Referring to FIG. 10, an air flow sensor 70 is installed in the mounting member 400. When in use, the outside air enters the outer casing 200 through an air hole (not shown) of the mounting member 400, the air flow passes through the air flow sensor 70, and then the air flow sensor 70 is activated. The air flow sensor 70 transmits information to a controller (not shown), and the controller controls the power supply assembly 300 to supply power to the heating member 11. Next, the air flow flows into the air ducts 26 through a gap between the outer casing 200 and the power supply assembly 300. And then, the aerosol formed by heating and atomizing the liquid atomizable substance by the heating member 11 passes through the air passage 13 and the suction nozzle 17 with the air flow for the user to inhale.

Referring to FIGS. 9 and 10, a protective cover 500 is detachably sleeved on an end of the outer casing 200 away from the suction nozzle 17. When not in use, the protective cover 500 covers the end of the outer casing 200 to keep out external contaminants entering the outer casing 200 through the mounting member 400, thereby protecting the power supply assembly 300 in the outer casing 200.

Referring to FIGS. 1 to 10, an embodiment of a method of assembling the inhalation device 1000 is provided. The method includes the following steps S10 to S50:

S10, assembling the atomizing assembly 10 using the following steps S11 to S13:

S11, referring to FIG. 3, installing the heating member 11 in the first through hole 121 of the liquid guiding member 12, the heating member 11 abuts against the inner wall of the first through hole 121 of the liquid guiding member 12, and installing the liquid guiding member 12 equipped with the heating member 11 in the housing 142.

Specifically, the net-like heating member is sleeved on a cylindrical iron rod (not shown), and the iron rod sleeved with the net-like heating member is inserted into the first through hole 121 of the liquid guiding member 12, the net-like heating member abuts against the inner wall of the first through hole 121. The liquid guiding member 12 equipped with the net-like heating member can be quickly installed into the core shell 14 through the two slots 1423 of the core shell 14.

S12, referring to FIGS. 3, 5, and 6, installing the core shell 14 obtained in step S11 in the liquid guiding pipe 15, and installing the second end 152 of the liquid guiding pipe 15 on the sealing member 20.

Specifically, the core shell 14 with the iron rod is inserted into the liquid guiding pipe 15, then the second end 152 of the liquid guiding pipe 15 is installed on the sealing member 20 equipped with the first sealing body 30, and the iron rod is removed. It is to be understood, the first sealing body 30 is installed in the vent 25 of the sealing member 20, the bottom of the first sealing body 30 abuts against the flange 29, part of the base 141 in the core shell 14 extends out of the liquid guiding pipe 15. When the liquid guiding pipe 15 is mounted on the first sealing body 30, the clamping blocks 34 are clamped by the clamping notches 1411. The liquid guiding pipe 15 presses tightly against the threads on the inner wall of the vent 25 to seal the connection between the liquid guiding pipe 15 and the sealing member 20.

S13, referring to FIG. 3, sleeving the second sealing body 16 on the first end 151 of the liquid guiding pipe 15, to obtain the atomizing assembly 10.

S20, referring to FIGS. 5 to 7, installing the atomizing assembly 10 in the liquid storage pipe 40, forming the liquid storage chamber 41 for storing the liquid atomizable substance in the liquid storage pipe 40, and injecting the liquid atomizable substance into the liquid storage chamber 41, to obtain the atomizer 100.

Specifically, the alignment protrusions 28 on the sealing member 20 are aligned with the alignment notches 42 on the liquid storage pipe 40, so that the mounting protrusions 27 of the sealing member 20 are accurately installed in the mounting holes 43 of the liquid storage pipe 40. The second sealing body 16 on the liquid guiding pipe 15 is also aligned with the air guiding pipe 44 in the liquid storage pipe 40, so that the second sealing body 16 and the sealing member 20 seal the gap between the atomizing assembly 10 and the liquid storage pipe 40, and the liquid storage chamber 41 is formed in the liquid storage pipe 40. When injecting liquid into the liquid storage chamber 41, the end of the liquid storage pipe 40 with the sealing member 20 is placed upward, the plug 50 in the sealing member 20 is removed, and the liquid is injected into the liquid storage chamber 41 through the liquid injection port 80 through the liquid injection gun or other liquid injection mechanism. After the injection, the plug 50 is re-inserted into the liquid injection port 80, so that the liquid storage chamber 41 becomes a closed chamber.

S30, referring to FIGS. 9 and 10, installing the air flow sensor 70 in the mounting member 400 and electrically connecting the air flow sensor 70 with the power supply assembly 300.

Specifically, the air flow sensor 70 is a microphone, pins of the microphone (not shown) pass through the mounting member 400, and the pins of the microphone are soldered to the power supply assembly 300. The power supply assembly 300 is a cylindrical battery. In some embodiments, the pins of the microphone and solder joints of the power supply assembly 300 are wrapped with a heat-resisting adhesive tape.

S40, referring to FIGS. 9 and 10, installing the liquid absorbent cotton 60 on the atomizer 100 and electrically connecting the heating member 11 with the air flow sensor 70.

Specifically, the liquid absorbent cotton 60 is installed on the sealing member 20, two pins (not shown) of the heating member 11 extend through the liquid absorbent cotton 60 respectively, and the two pins of the heating member 11 are respectively connected with two pins of the microphone by soldering, so as to realize the electrical connection between the heating member 11 and the air flow sensor 70. When in use, the air flow sensor 70 is started by the air, the controller (not shown) controls the power supply assembly 300 to supply power to the heating member 11, the heating member 11 heats and atomizes the liquid atomizable substance in the liquid guiding member 12 to obtain the aerosol, and the aerosol passes through the air passage 13 for the user to inhale.

S50, referring to FIGS. 9 and 10, installing the atomizer 100 and the power supply assembly 300 in the outer casing 200, and installing the mounting member 400 on the end of the outer casing 200 away from the suction nozzle 17, to obtain the inhalation device 1000.

Specifically, before the atomizer 100 is installed in the outer casing 200, a protective film (not shown) wrapped around the connection between the suction nozzle 17 and the liquid storage pipe 40 is tore off. The protective film can reduce the pollution of external dust to the suction nozzle 17 during the assembly of the inhalation device 1000. The air flow sensor 70, the power supply assembly 300, and the atomizer 100 are installed into the outer casing 200 in such order, so that an end of the outer casing 200 abuts against the bulge loop 172 of the suction nozzle 17. During the installing process, the outer wall of the liquid storage pipe 40 is coated with adhesive near the bulge loop 172 in advance to fix the outer casing 200 on the atomizer 100. The dust plug 18 is installed in the suction port 171 of the suction nozzle 17 with a hand-punch. Finally, the mounting member 400 is installed at an end of the outer casing 200 to obtain the inhalation device 1000.

Referring to FIGS. 9 and 10, in order to prevent external dust from entering the inhalation device 1000, the protective cover 500 is buckled on the outer casing 200 and covers the end of the outer casing 200 away from the suction nozzle 17.

### Second Embodiment

A difference between the second embodiment and first embodiment is the structure of the sealing rings.

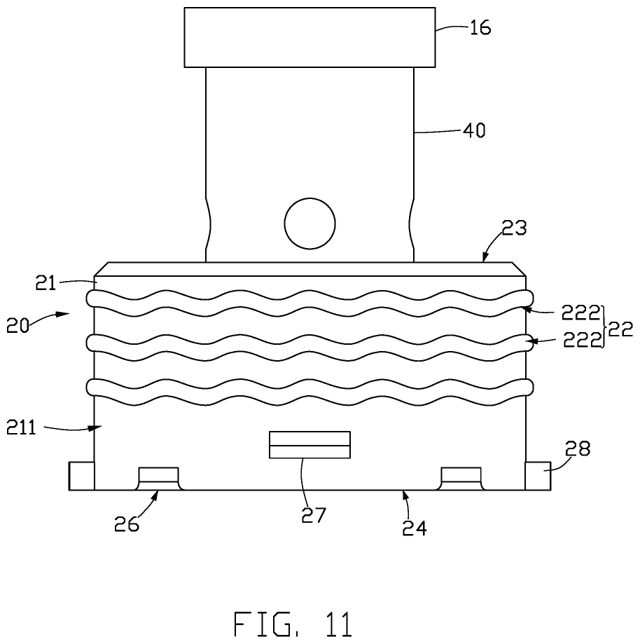

Referring to FIG. 11, in the second embodiment, a positive projection of the sealing ring 222 on the outer wall 211 is wavy. This arrangement can increase a contact area between the sealing ring 222 and the inner wall of the liquid storage pipe 40, so as to further improve the tightness of the sealing member 20 sealing the end of the liquid storage pipe 40.

### Third Embodiment

A difference between the third embodiment and first embodiment is the structure of the sealing rings.

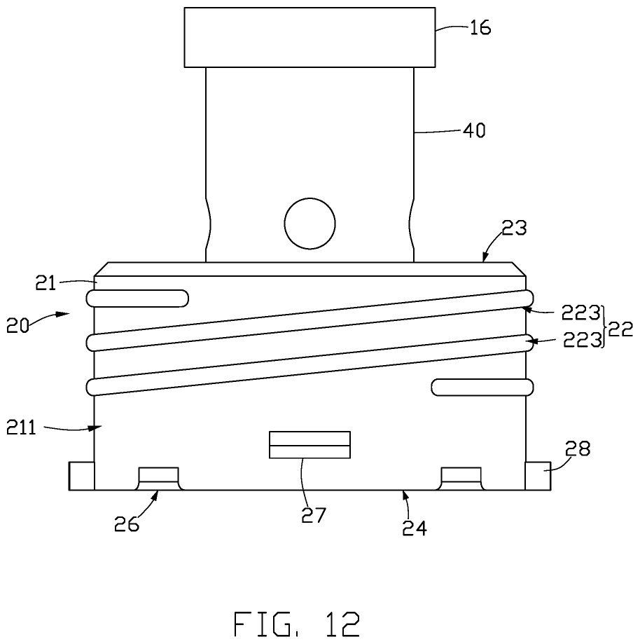

Referring to FIG. 12, in the third embodiment, the sealing rings 223 form a threaded resisting portion 22. By increasing the contact area between the resisting portion 22 and the liquid storage pipe 40, the tightness of the sealing member 20 sealing the end of the liquid storage pipe 40 is further improved.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A sealing member for sealing an end of a liquid storage pipe of an atomizer, the sealing member comprising:
a base body comprising a first surface, a second surface opposite to the first surface, and an outer wall connecting the first surface with the second surface, the base body defining a vent passing through the first surface and the second surface; and
a resisting portion disposed on the outer wall, wherein the resisting portion is configured to be compressed against an inner wall of the liquid storage pipe.

2. The sealing member of claim 1, wherein the resisting portion comprises a plurality of sealing rings disposed annularly on the outer wall.

3. The sealing member of claim 1, wherein the second surface defines a plurality of air ducts communicating with the vent.

4. The sealing member of claim 1, wherein the base body defines a liquid injection port apart from the vent, a plug is movably disposed in the liquid injection port.

5. An atomizer comprising:
a liquid storage pipe;
an atomizing assembly accommodated in the liquid storage pipe; and
a sealing member disposed on an end of the liquid storage pipe, the sealing member comprising:
a base body comprising a first surface, a second surface opposite to the first surface, and an outer wall connecting the first surface and the second surface, the base body defining a vent passing through the first surface and the second surface; and
a resisting portion disposed on the outer wall, wherein the resisting portion is compressed against an inner wall of the liquid storage pipe.

6. The atomizer of claim 5, wherein a mounting protrusion is disposed on the outer wall, the mounting protrusion is close to the second surface, the inner wall of the liquid storage pipe defines a mounting hole, the mounting protrusion is disposed in the mounting hole.

7. The atomizer of claim 5, wherein an alignment protrusion is disposed on the outer wall, the inner wall of the liquid storage pipe defines an alignment notch, the alignment protrusion is disposed in the alignment notch.

8. The atomizer of claim 5, further comprising a first sealing body defining a through hole, wherein an inner wall of the vent extends towards a central axis of the vent to form a flange, the first sealing body extends in a central axis of the through hole to form a guide pipe.

9. The atomizer of claim 8, further comprising a core shell and a heating member, wherein the core shell defines a receiving cavity passing through the core shell, the heating member is disposed in the receiving cavity, and the core shell is sleeved on the first sealing body and extends out of the vent of the sealing member.

10. The atomizer of claim 9, further comprising a liquid guiding pipe defining a plurality of liquid guiding holes, wherein the core shell is accommodated in the liquid guiding pipe, and part of the plurality of liquid guiding holes is placed in the vent and is in contact with the inner wall of the vent.

11. The atomizer of claim 10, wherein an air guiding pipe and a second sealing body are disposed in the liquid storage pipe, the liquid guiding pipe communicates with the air guiding pipe, and the second sealing body is hermetically connected at a connection between the liquid guiding pipe and the air guiding pipe.

12. The atomizer of claim 5, wherein the resisting portion comprises a plurality of sealing rings disposed annularly on the outer wall.

13. The atomizer of claim 5, wherein the second surface defines a plurality of air ducts communicating with the vent.

14. The atomizer of claim 5, wherein the base body defines a liquid injection port apart from the vent, a plug is movably disposed in the liquid injection port.

15. An inhalation device comprising:
an outer casing; and
an atomizer disposed in the outer casing, the atomizer comprising:
a liquid storage pipe;
an atomizing assembly accommodated in the liquid storage pipe; and
a sealing member disposed on an end of the liquid storage pipe, the sealing member comprising:
a base body comprising a first surface, a second surface opposite to the first surface, and an outer wall connecting the first surface and the second surface, the base body defining a vent passing through the first surface and the second surface; and
a resisting portion disposed on the outer wall, wherein the resisting portion is compressed against an inner wall of the liquid storage pipe.

16. The inhalation device of claim 15, wherein a mounting protrusion is disposed on the outer wall, the mounting protrusion is close to the second surface, the inner wall of the liquid storage pipe defines a mounting hole, the mounting protrusion is disposed in the mounting hole.

17. The inhalation device of claim 15, wherein an alignment protrusion is disposed on the outer wall, the inner wall of the liquid storage pipe defines an alignment notch, the alignment protrusion is disposed in the alignment notch.

18. The inhalation device of claim 15, wherein the atomizer further comprises a first sealing body defining a through hole, an inner wall of the vent extends towards a central axis of the vent to form a flange, the first sealing body extends in a central axis of the through hole to form a guide pipe.

19. The inhalation device of claim 18, wherein the atomizer further comprises a core shell and a heating member, the core shell defines a receiving cavity passing through the core shell, the heating member is disposed in the receiving cavity, and the core shell is sleeved on the first sealing body and extends out of the vent of the sealing member.

20. The inhalation device of claim 19, wherein the atomizer further comprises a liquid guiding pipe defining a plurality of liquid guiding holes, the core shell is accommodated in the liquid guiding pipe, and part of the plurality of liquid guiding holes is placed in the vent and is in contact with the inner wall of the vent.
